(19) United States
(12) Patent Application Publication
(10) Pub. No.: US 2018/0028396 A1
(43) Pub. Date: Feb. 1, 2018

(54) DEVICE AND METHODS FOR MASSAGE, ACUPRESSURE MYOFASCIAL RELEASE AND TRIGGER POINT THERAPY

(71) Applicant: THE TRUSTEES OF COLUMBIA UNIVERSITY IN THE CITY OF NEW YORK, NEW YORK, NY (US)

(72) Inventor: Marc Brodsky, Stamford, CT (US)

(21) Appl. No.: 15/458,163

(22) Filed: Mar. 14, 2017

Related U.S. Application Data
(63) Continuation of application No. 13/633,117, filed on Oct. 1, 2012, now abandoned.
(60) Provisional application No. 61/541,470, filed on Sep. 30, 2011.

(51) Int. Cl. A61H 15/00; A61H 39/04; A61N 5/06; A61H 23/02; A61N 1/32

(52) U.S. Cl. CPC A61H 15/00; A61N 5/0619; A61H 23/0236; A61H 23/0245; A61H 23/0263; A61H 39/04; A61H 15/0092; A61H 2201/0257; A61H 2201/0214; A61H 2201/0207; A61H 2201/10; A61N 2005/0659; A61H 2201/1253; A61N 1/322; A61H 2015/0042; A61H 2201/102

(57) ABSTRACT

A device for self-administration of massage, trigger point therapy and/or myofascial release, and methods for same.

### CROSS-REFERENCE TO RELATED APPLICATIONS

This application claims priority to U.S. Provisional Application No. 61/541,470, filed Sep. 30, 2011, which is hereby incorporated by reference in its entirety.

### FIELD

The disclosed subject matter generally relates to devices and methods for providing massage, trigger point therapy and/or reducing myofascial pain and fibromyalgia in a subject body.

### BACKGROUND

Many people suffer from chronic muscular pain. In many of these cases, the cause of the pain is not known and the patients rely on long-term symptomatic treatment. For instance, fibromyalgia is medically unexplained syndrome characterized by chronic widespread pain and a heightened and painful response to pressure. Other core symptoms include debilitating fatigue, sleep disturbance, and joint stiffness. Fibromyalgia affects 2-4% of the population and its cause is unknown. No cure exists. Many of these patients resort to alternative medicine for pain management, including acupressure and trigger point therapy. However, such therapies are often costly and time consuming.

Likewise, there are also many people that suffer from myofascial pain. Fascia is specialized connective tissue provides support and protection of the body. The fascia surrounds the muscles, bones and joints and sometimes becomes restricted due to psychogenic disease, overuse, trauma, infectious agents, or inactivity. When fascia becomes restricted it can result in, pain, muscle tension, and diminished blood flow. Myofascial release is a technique in which massage or acupressure is used to release tight fascia. Often the technique is used by rolling the body on an elongate cylindrical foam roller. The pressure on the fascia can break down the restrictions and promote normal soft-tissue extensibility. This technique also improves flexibility, function, performance and can reduce injuries.

Trigger point therapy relieves areas of muscle that are painful to palpation and can sometimes become thick, tough and knotted. Therapy includes applying pressure to the trigger points, e.g., paraspinal muscles for upper and lower back pain and gluteus muscle for hip pain can relieve the muscle and promote relaxation and reduce pain.

Massage therapy has existed for many years. A variety of massage therapies have become popular, including acupressure, cranio-sacral, deep tissue, and Swedish therapies. Additional therapies include myofascial release, reflexology, shiatsu, trigger point therapy, and hot stone therapy.

Massage is a very effective technique for controlling pain, and promoting body health. Massage has been known to promote release of the body's natural painkillers by stimulating release of endorphins and provide deep relaxation by relieving muscle tension, spasm, and stiffness—all pain contributors. More currently, massage techniques such as trigger point massage therapy and myofascial release have been designed to alleviate trigger point and myofascial pain through cycles of isolated pressure and release.

Trigger point therapy is a massage technique that involves finding “trigger points” in the body and applying pressure to relieve symptoms of muscular pain and discomfort. A trigger point may also include a tight and tender spot in a muscle that refers pain (or “triggers” pain) to other areas of the body. Likewise, myofascial release is a massage therapy that involves finding tightened fascia in the body and applying pressure to alleviate the tension and discomfort. By applying pressure at the trigger point or the fascia, as the case may be, the trigger point or fascia is thereby “released.” The therapeutic results include reduction in pain and discomfort, and increased flexibility and overall promotion of health, well-being and fitness. A need exists for a device that can precisely provide massage and therapy to the trigger points and tightened fascia in a subject body.

### SUMMARY

The disclosed subject matter generally relates to devices and methods for providing massage, and more particularly to a self-operable device and methods for providing acupressure and trigger point therapy to a subject body. The massage device in accordance with the subject matter may include a tubular sleeve having at least a section configured to receive first and second rollers. The rollers are selectively supported in the sleeve such that a spaced distance can be maintained.

In one aspect, the disclosed subject matter provides a device suitable for self-administration of massage, acupressure, trigger point therapy and/or myofascial release. The device is easy to use, and facilitates accurate location and application of pressure at selected trigger points long the human body. The device generally includes a sleeve having a first section, second section, and a middle section. At least one section of the sleeve is enclosed and contains first and second rollers within the enclosure. The first and second rollers are be disposed in a side by side arrangement in the middle compartment of the device. The first and second rollers can have surfaces that are spaced apart from one another by a distance. The farthest distance permitted between the rollers is maintained by retainers. In this manner, the restrainer prevents substantial movement of the first and second rollers to another section of the sleeve, but also limits the furthermost distance permitted between the rollers. In an alternative embodiment, when a user of the device holds the sleeve ends, or handles, in each hand and places the rollers between the back and the wall to press hard-to-reach pressure points, the compartment that holds the rollers will be able to stretch for a spacing of 1.5 to 5.5 inches between the two 2.5 inch rollers. This distance between the outer surface of each roller (curve to curve) is commensurate with the relevant acupuncture points and trigger points of the body (see FIGS. 3 and 4 where many different trigger points are shown) that lie between 2 and 4 inches on each side of the spine. The flexible sleeve will allow a distance of rollers between 1.5 and 5.5 inches that allow for customized treatment, since the relevant acupuncture points and trigger points lie 2 to 4 inches on either side from the midline. In some embodiments, for example when the device is not in use the first and second rollers are separated by a distance of less than one inch. For example, the first and second rollers are touching.

In one embodiment, the first and second rollers consist of only two rollers. However, in alternate embodiments, the first and second rollers can comprise a plurality of rollers. In some embodiments, the first and second rollers comprise foam, rubber, latex, or sponge material. The material should have a sufficient density to provide pressure to the trigger point of the body when depressed for example by an exerting force. In some embodiments, the rollers each have a diameter of less than five inches. For example, a diameter of about 2.5 plus or minus 0.5 inches. The first and second rollers have an outer surface that may be smooth or textured. In the case of textured surfaces, the texture can include ridges, rounded protrusions, nodes etc. The rollers can include an energy source, such as a battery, to vibrate or emit heat.

The sleeve can be formed from various materials, such as elastic, nylon, cotton, velour, or any other cloth or fabric. In some embodiments, the ends of the sleeve body include a gripping member. The gripping member can be as simple as a rope, or can be a molded handle.

In another aspect, a kit is provided that comprises the massage device and instructions for use. The instructional material includes methods of using the device to achieve a variety of therapies or treatments. For example and not limitation, the device can be used massage, trigger point therapy release, myofascial release, and pain alleviation from for example fibromyalgia and other syndromes.

Thus, in another aspect, a method of promoting physical health is provided. The method includes placing the massage device between the body and a rigid surface. The first and second rollers enclosed in the sleeve are positioned at or near trigger points or tightened fascia. Localized pressure is exerted on the trigger points or the fascia manipulating the first and second rollers between the rigid surface and the body. The rigid surface can be a wall, a floor or a hand of a second person. The method can achieve trigger point therapy, myofascial release or deep tissue massage. Additionally, the method can alleviate pain from a variety of ailments including fibromyalgia.

### BRIEF DESCRIPTION OF DRAWINGS

FIG. 1 is a schematic representation of a device in accordance with the disclosed subject matter.

FIG. 2 is a schematic representation of a device in accordance with the disclosed subject matter.

FIGS. 3A-3D provide a schematic representation of various trigger points in the upper body that can be treated using the device and method of the disclosed subject matter.

FIGS. 4A-4C provide a schematic representation of various trigger points in the lower body that can be treated using the device and method of the disclosed subject matter.

FIGS. 5A-5C provide a schematic representation of the interior of devices in accordance with embodiments of the disclosed subject matter.

FIG. 6 is a schematic representation of the interior of a device in accordance with an embodiment of the disclosed subject matter.

FIGS. 7A-7D provide a schematic representation of releasable fasters of a device in accordance with embodiment of the disclosed subject matter.

### DETAILED DESCRIPTION OF EXEMPLARY EMBODIMENTS

In accordance with one aspect of the disclosed subject matter, a device is provided for providing massage, trigger point therapy or myofascial release to a subject body. The device can be self-operated to provide self administration of the therapy techniques or can be used by a second person to provide therapy techniques to the subject body. The device is configured to easily target the trigger points for massage, acupressure, trigger point therapy and myofascial release. The disclosed subject matter also relates to methods for promoting physical health by self-administration of massage, acupressure, trigger point therapy and myofascial release.

### The Massage Device

In one aspect of the disclosed subject matter, a massage device is provided. The device generally includes a tubular sleeve and at least two rollers within a portion of the sleeve. The two rollers are spaced at a distance and maintained at a distance to promote targeting of the trigger points and fascia of a subject body so that the massage techniques can be more precisely administered to the body for more effective results.

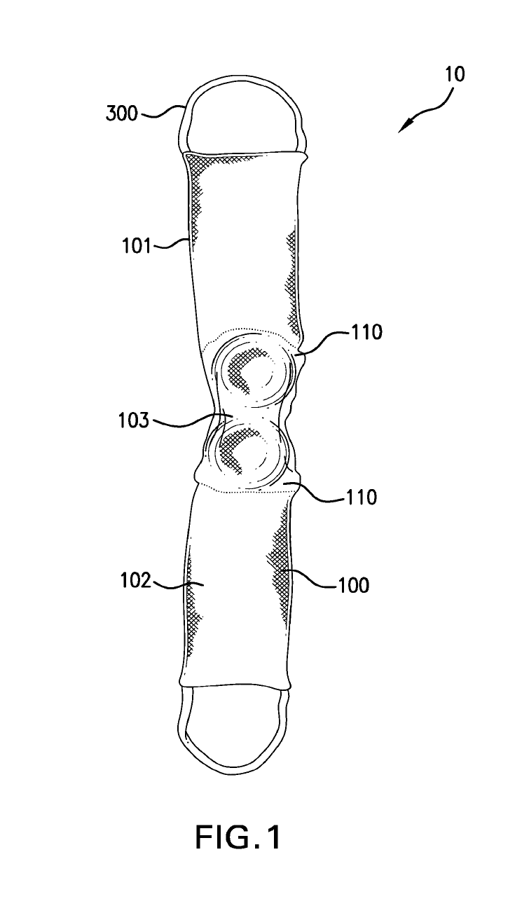

In one embodiment, the massage device 10, as illustrated in FIG. 1, includes a generally tubular sleeve 100 having three sections: a first section 101, second section 102 and a middle section 103. However, the sleeve can be configured with more or less sections, if desired. The sleeve 100 generally has a length of about 20 to about 36 inches, e.g., 22 inches. For the purpose of illustration and not limitation, the sleeve 100 can be formed from material that is a fabric or cloth, including but not limited to elastic, cotton, or velour. Thus, the sleeve 100 can be substantially foldable and can be stretchable.

In one embodiment, for example, the first and second sections (101 and 102) each have a length of about 5 to 12 inches, and the middle section 103 has a length of about 6 to 10 inches while the fabric or cloth is in the resting state. For example, in one embodiment, the first (101) and second (102) sections have a length of about 8 inches, and the middle section 103 containing the roller has a length of about 6 inches. When stretched, however, the length of the sections can lengthen considerably. For example, a 12-inch section can lengthen to about 20 to 24 inches, and a 6 inch middle section can lengthen to about 8 to 10 inches.

The device 10 can further include handles 300 disposed at opposite ends of sleeve 100. The handles 300 can comprise any type of gripping member including foam, cord, fabric, or wooden or polymeric handle. The handle 300 may be at least partially rounded or ergonomic to fit within the subject's hand. For example, the handle 300 may include an ergonomic contours with finger receiving depressions. In some embodiments, the handle 300 can be a wooden or polymeric handle covered with resilient and/or elastomeric material for increased comfort and to enable more secure gripping.

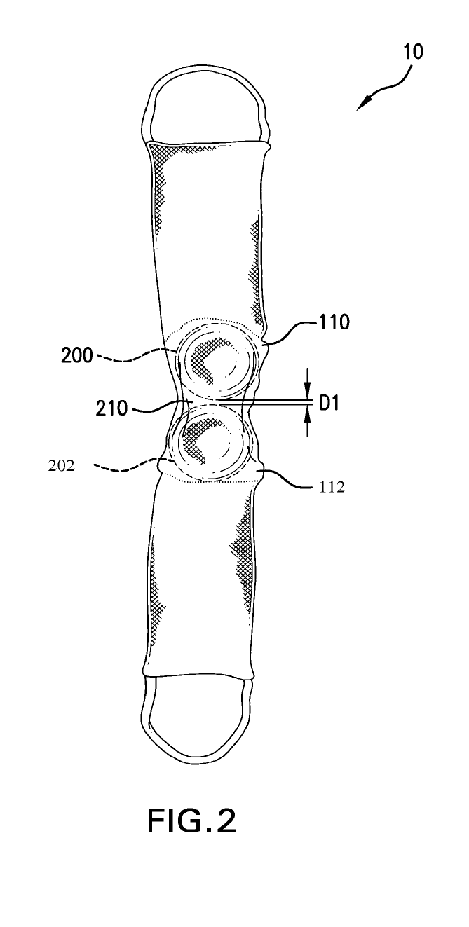

Referring to FIG. 2, the sleeve 100 includes first and second rollers 200, 202 within the tubular body of the sleeve 100. In one embodiment, the first and second rollers 200, 202 are enclosed within the middle section 103 of the sleeve 100. The middle section 103 is separated from the first and second sections 101, 102, of the sleeve 100 and defines an enclosed or substantially enclosed envelope to receive the first and second rollers 200, 202. The enclosure can be created by restrainers 110, 112 disposed at both ends of the middle section 103. In one embodiment, the restrainers 110 and 112 can include threaded seams sewn into the material of the sleeve 100. However, other methods of enclosing the section 103 can be employed. The restrainers 110 serve to section off the middle section 103 from the other portions of the sleeve 100, and more importantly, to maintain the first and second rollers 200, 202 within a particular distance (D1) from each other. The spacing between the seams sewn into the sleeve in the middle section 103 is about 6 inches. However when the device is in use, the space can elongate to about 9 to 10 inches.

The first and second rollers 200, 202 are spaced apart at a distance of between 0.25 and 3 inches (depending on whether the sleeve is stretched) within the section 103 of the sleeve 100. In some embodiments, a distance of less than 3 inches is defined between the first and second rollers 200, 202. The distance between the first and second rollers 200, 202 is maintained by the restrainers 110, 112. In other words, the farthest distance possible between the first and second rollers is limited and maintained by the restrainers 110, 112. In some embodiments, the first and second rollers 200, 202 are spaced apart at a distance adapted to apply pressure on both sides of a spinous process, which may be between 2 and 4 inches in an adult human.

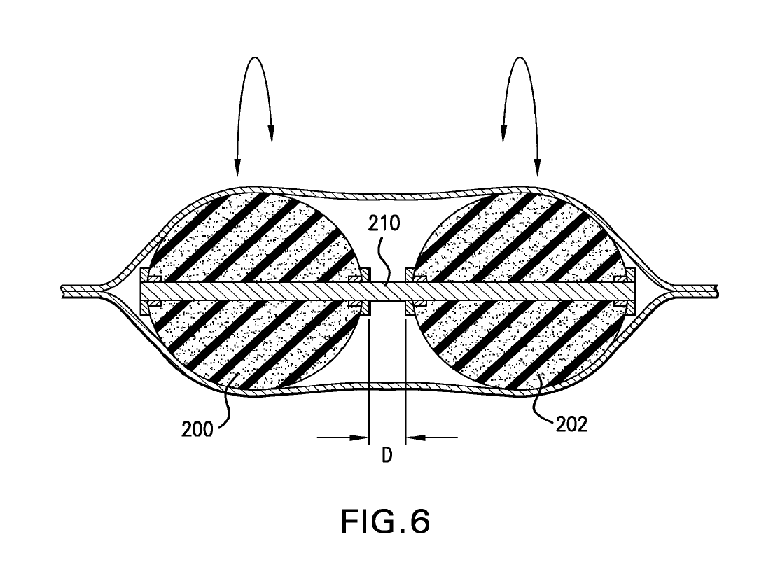

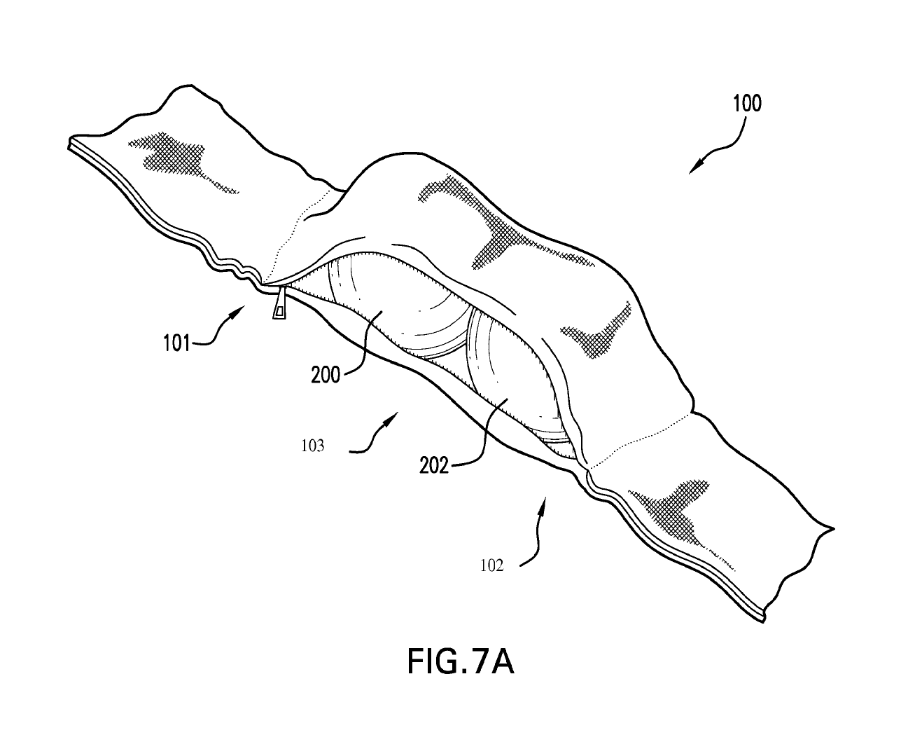

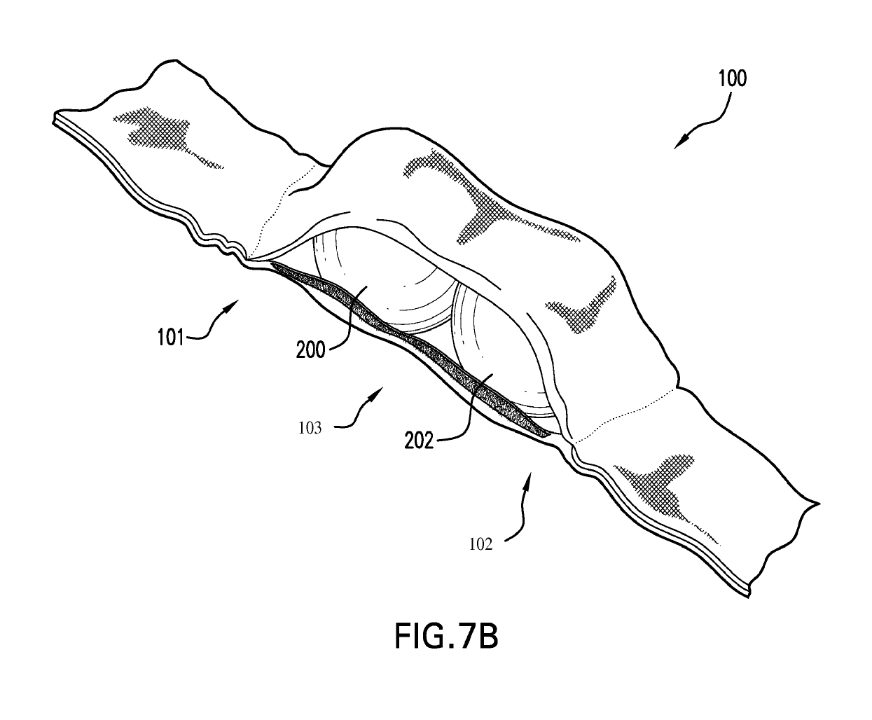

Referring to FIG. 6, in some embodiments, the first and second rollers 200, 202 can be interconnected by an interconnector 210 that maintains a constant determined space between the first and second rollers 200, 202. In this regard, however, the interconnector 210 does not render the rollers 200, 202 stationary. Instead, the rollers 200, 202 are configured to rotate about the interconnector 210. In this manner, the interconnector 210 can include a socket for receiving both the first roller 200 and the second roller 202. The rollers 200, 202 are uninhibited by the interconnector 210. In one embodiment, the rollers 200, 202 may include a bore through the diameter of the body of the roller. The socket can include an axle that extends through the diameter of the rollers 200, 202 so that the rollers can move about the body of the subject.

In some embodiments, the first and second rollers 200, 202 have a diameter of less than 5 inches. For example, and not limitation the diameter is about 2.25 to 2.5 inches. In other embodiments, the first and second rollers 200, 202 abut each other. The rollers 200, 202 are configured and formed of a material suitable to apply localized pressure to the fascia or trigger points for a period of time. The first and second rollers can be made from a variety of materials including foam, sponge, latex and the like. The rollers 200, 202 can be spherical or cylindrical in shape and the outer surface of the rollers can be smooth or textured. For example, the texture can include rounded bumps or protrusions, nodes, spikes, and the like. The textured surface may increase the surface area of the rollers in contact with the trigger point or fascia during operation.

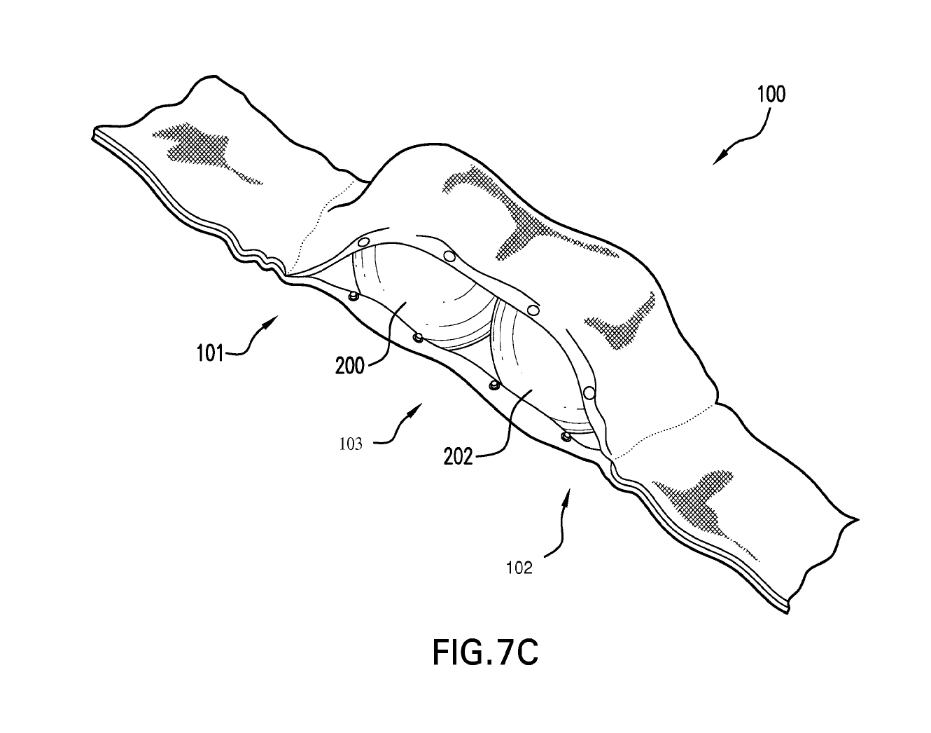

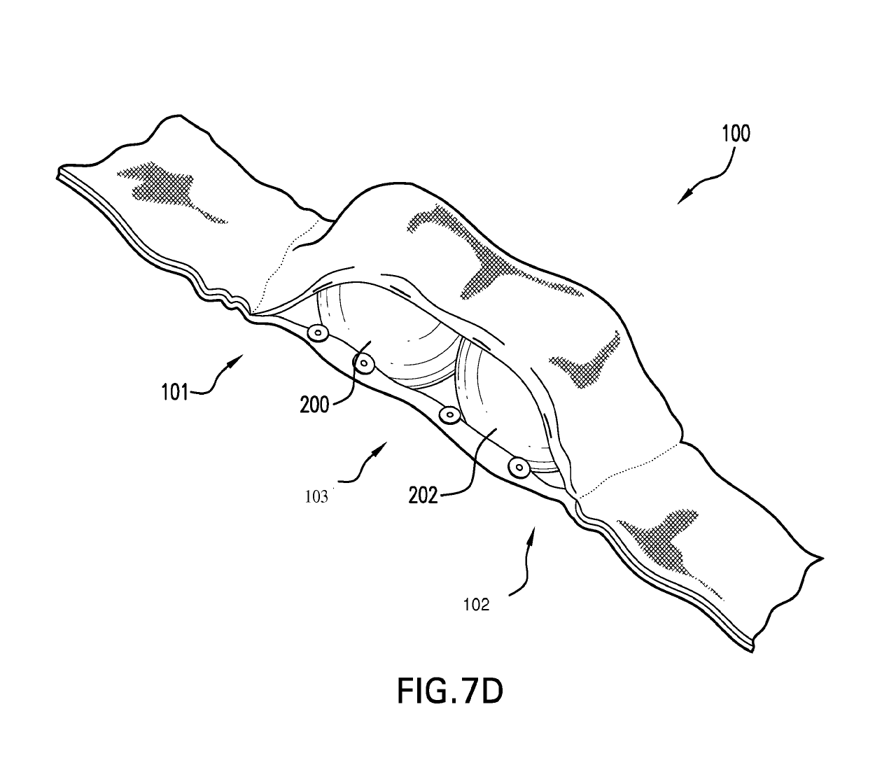

Referring to FIG. 7, in some embodiments, sleeve 100 may include a releasable fastener such as a zipper (FIG. 7A) or the like which is disposed in at least one of the first 101, second 102 or middle sections 103. The fastener can be opened to gain access to one or more of the sections (101, 102 or 103), for example, the section with the rollers 200, 202. In this manner, an enclosed section can be opened to replace or insert different or additional rollers 200, 202. The releasable fastener includes but is not limited to: slide fastener components, snaps (FIG. 7C), buttons (FIG. 7D), hook and loop fastening systems i.e. VELCRO™ (FIG. 7B), straps, ties and the like.

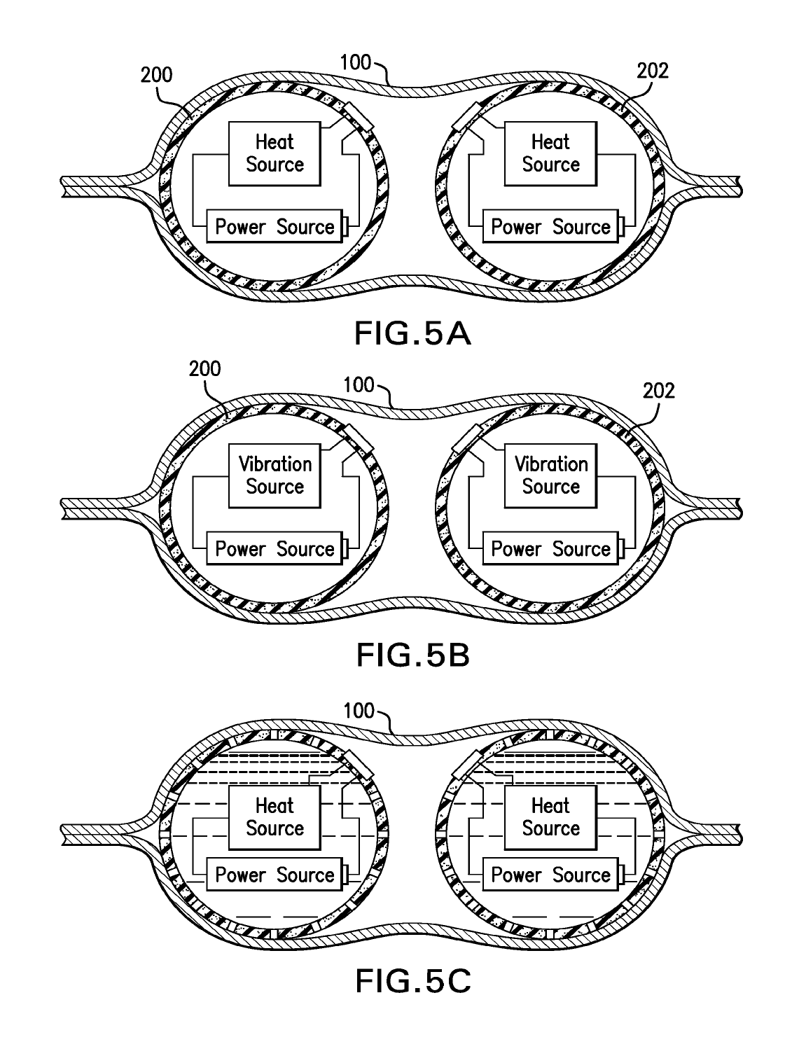

Referring to FIG. 5, The first and second rollers 200, 202 can be configured to vibrate and/or emit heat. The rollers 200, 202 can be configured with an internal or external energy source to provide the power to cause vibration or heat. For example, the rollers 200, 202 can include a defined core for accommodating heating, vibrating or massage liquid dispensing assemblies. In this regard, the rollers 200, 202 can incorporate a heat source for providing heat to a subject (FIG. 5A). The rollers 200, 202 as noted above can comprise a material such as sponge or foam. When moistened, the rollers 200, 202 can the heat source can provide the subject with a moist source of heat. Alternatively, the rollers 200, 202 may be heated prior to installation in the sleeve 100 so that the massage may include application of heat in addition to more or less penetrating strokes.

The rollers 200, 202 can include an internal vibrator that is battery activated and controlled with a switch located on the rollers (FIG. 5B). The vibrator can be driven by receiving power from a power source such as a battery. The vibrations of the vibrator can be transmitted to the rollers and imparted to the body of the subject. The vibrator, for example, can include a control unit having a motor that spins an unbalanced gear/weight combination at for example 100 to 500 RPM causing vibration of the rollers.

The first and second rollers 200, 202 can be configured to emit cold. For example, the rollers can include a defined core for accommodating a cold element. The cold element can comprise enclosed ice, or another suitable material known in the art for retaining cold. Alternatively, the rollers 200, 202 may be cooled prior to installation in the sleeve 100 so that the massage may include application of cold in addition to more or less penetrating strokes.

Additionally, massage oils may be applied to the person's skin by the liquid dispensing assembly. The liquid can be heated by the heating unit before application to the skin. In this manner, the rollers 200, 202 may include pores or depot to receive aromatic or non-aromatic oils. The pressure exerted on the rollers from the subject body during use can operate to release the oils from the rollers. Applying oils in this manner can provide lubrication of the rollers and the subject's body. In this regard, the rollers 200, 202 can be covered with an absorbable material enclosed in a washable cover material. The massage oil can include a topical medication. Alternatively, a non-oil based topical medication can be substituted for the massage oil.

The first and second rollers 200, 202 can be configured to provide additional means of stimulation. For example, the rollers can include a defined core for accommodating an emitter of infrared light, a power source for electrical transcutaneous stimulation, an emitter adapted to provide controlled release of an aromatic compound, a speaker adapted to provide sound, or an ultrasonic emitter.

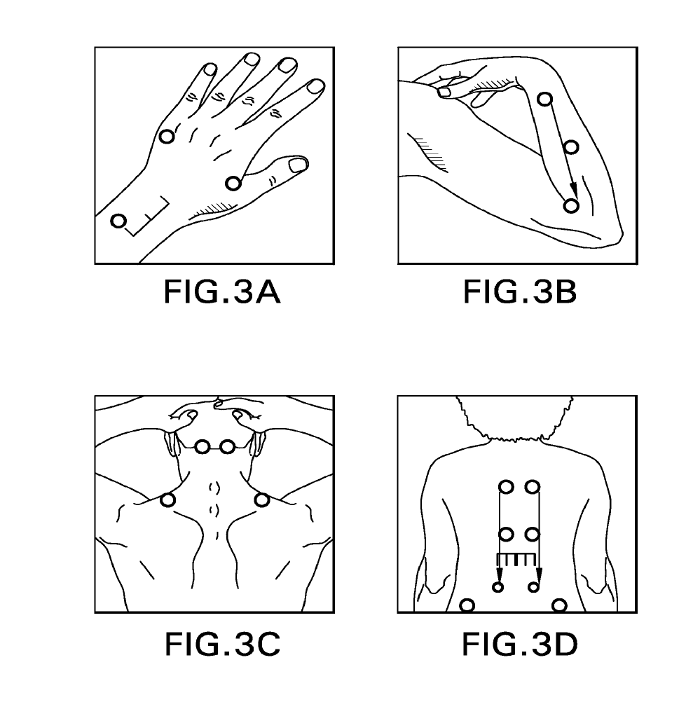

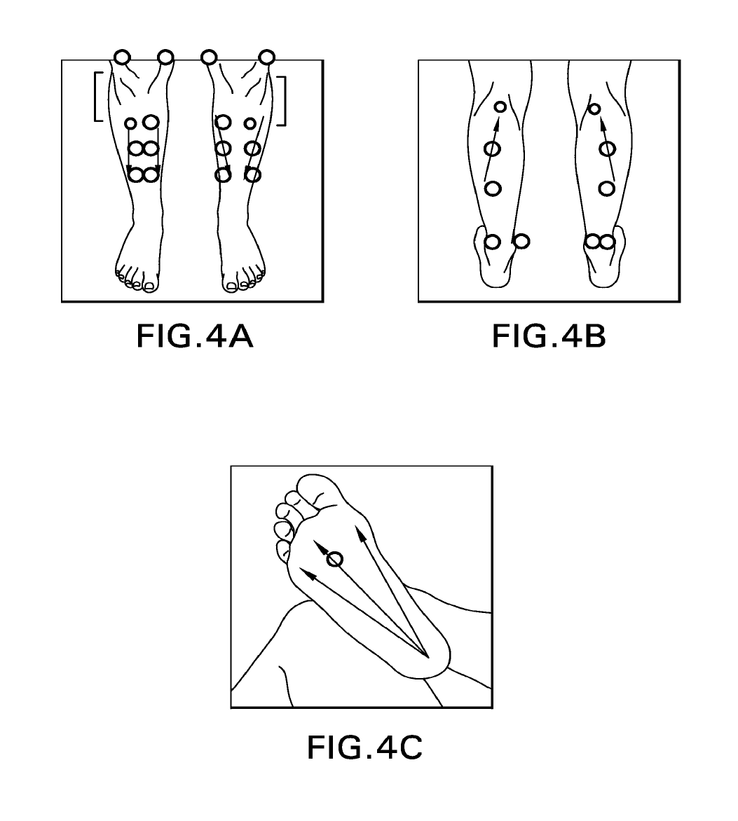

In another aspect, a method for promoting physical well-being is provided. According to the method, a person rolls the device 10 along areas of discomfort or known trigger point or tightened fascia locations. Referring to FIGS. 3 and 4, various trigger points are schematically illustrated. In accordance with the method, the device 10 comprising first and second rollers 200, 202 is placed between the body and a rigid surface. The first and second rollers 200, 202 are positioned at or near one or more trigger points or tightened fascia and contact the rigid surface and the body at a particular force so as to apply localized pressure to the trigger point or fascia. The rigid surface may be a wall, a floor or even the hand of a second person. Thus the device 10 can be self-operable to perform the method or a second person can administer the method.

The method can be used to achieve trigger point therapy, massage, acupressure, or myofascial release. In some embodiments, a person can position the device 10 between himself and a rigid surface and roll the device along hard to reach areas, such as the back and shoulders, to find trigger points. When a trigger point is found, the person manipulates the device to apply increased pressure for a period of time, e.g., 30 seconds to 3 minutes, until the trigger point releases. Applying the localized pressure to the body also can achieve alleviation of pain and discomfort, such as the pain and/or discomfort attributable to fibromyalgia, myofascial pain, headache, temporomandibular joint (jaw) pain, arthritis, muscle tension, and other syndromes. As the device 10 rolls, the rollers 200, 202 provide incremental pressure along the body, thereby soothing tight or tender spots in the muscle.

For example, in one embodiment, referring to FIG. 3A, the method includes positioning the first and second rollers 200, 202 and depressing the two points on the hand for one deep slow breath for about 3-5 seconds each point. The user can start at the point that would sit above the face of a watch worn on the wrist, and press three points up the front of the forearm to the outside crease of the elbow, as shown in FIG. 3B. Each point can be pressed for one deep breath. The whole forearm treatment can take about 15 seconds.

Referring to FIG. 3C, the first and second rollers 200, 202 can be used to press the points on top of where the neck meets the shoulder for one deep slow breath, e.g., 3-5 seconds at each point. If desired, interlocked fingers can be used to press the thumbs into the tender points at the base of the skull for one deep slow breath, e.g., 3-5 seconds each point.

Further, the first and second rollers 200, 202 can be pressed against a rigid surface such as a wall, and using the weight of the body the rollers can be used to depress the trigger points along the back as shown in FIG. 3D. In one embodiment, the method includes positioning the first and second rollers 200, 202 at either side of a spinous process, and wrapping the tubular sleeve 100 around the body and outside of the arms in order to position the rollers 200, 202 at trigger points along the back. Referring to 4A, trigger points along the inside of the front of the leg just above the knee can be pressed for one deep slow breath, e.g., 3-5 seconds each point. The device 10 can be used to squeeze the muscle at the inner and outer sides of the shin bone. Referring to FIG. 4B, the Achilles tendon can be positioned between the first and second rollers and the rollers can be used to squeeze the Achilles tendon for one deep slow breath Referring to FIG. 4B, the rollers 200, 202 can be used to press the calf at three or more points to the back of the knee. To provide localized pressure to the bottom of the foot, the device 10 can be positioned between a floor or wall and the bottom side of the foot. Referring to FIG. 4C, the rollers 200, 202 can be rolled by the foot concentrating on the point at the roller of the foot for approximately 15 or more seconds. Thus, the device 10 can be used to work the entire body of an individual.

In another aspect, a kit is provided. The kit generally includes the device described above and an instruction for use of the device. In some embodiments, the instruction medium includes an instruction sheet, a DVD, or a CD. For example and not limitation, the instruction includes instruction for using the device to alleviate pain, perform message, trigger point therapy, acupressure, and/or myofascial release, or a combination of thereof.

In some embodiments, the kit further includes replaceable or additional rollers. The rollers can include the same or different dimensions than the first and second rollers 200, 202. For example, the first and second rollers 200, 202 can be spherical, and the replacement or additional rollers of the kit can be cylindrical. As yet another example, the first and second rollers 200, 202 can have a diameter of less than 3 inches, whereas the replacement or additional rollers may have a dimension larger or smaller than the first and second rollers 200, 202.

In some embodiments, correct location for pressure point stimulation on either side of the spine up and down the upper back, lower back, and hip areas can be accomplished by elastic means other than a sleeve. For example, an elastic band, string, spring, or other flexible member can be used. It will be apparent that the devices of the current disclosure can be used at locations in a variety of parts of the body, corresponding to trigger points (myofascial pain syndrome), tender points (fibromyalgia), and acupressure points (Chinese medicine), which are known in the art.

It is understood that the subject matter described herein is not limited to particular embodiments described, as such may, of course, vary. Nothing contained in the Abstract or the Summary should be understood as limiting the scope of the disclosure. It is also understood that the terminology used herein is for the purpose of describing particular embodiments only, and is not intended to be limiting. Where a range of values is provided, it is understood that each intervening value between the upper and lower limit of that range and any other stated or intervening value in that stated range, is encompassed within the disclosed subject matter.

Unless defined otherwise, all technical and scientific terms used herein have the same meaning as commonly understood by one of ordinary skill in the art to which this disclosed subject matter belongs. Although any methods and materials similar or equivalent to those described herein can also be used in the practice or testing of the present disclosed subject matter, this disclosure may specifically mention certain exemplary methods and materials. As used herein and in the appended claims, the singular forms “a,” “an,” and “the” include plural referents unless the context clearly dictates otherwise.

What is claimed is:

1. A device comprising:
a sleeve having a first section, second section, and a middle section therebetween;
first and second rollers disposed within a section of the sleeve, a spaced distance defined between the first roller and the second roller, and
a restrainer to prevent movement of the first and second rollers to another section of the sleeve.

2. The device of claim 1, wherein the first and second rollers consist of only two rollers.

3. The device of claim 1, wherein the first and second rollers comprise a plurality of rollers.

4. The device of claim 1, wherein the rollers are spherical.

5. The device of claim 1, wherein the rollers are cylindrical.

6. The device of claim 1, wherein the first and second rollers comprise foam.

7. The device of claim 6, wherein the first and second rollers comprise foam rubber.

8. The device of claim 1, wherein the first and second rollers comprise latex.

9. The device of claim 1, wherein the first and second rollers comprise sponge.

10. The device of claim 1, wherein the at least one of the first and second rollers has a diameter of less than three inches.

11. The device of claim 10, wherein both the first and second rollers has a diameter of less than 3 inches.

12. The device of claim 10, wherein both the first and rollers has a diameter of about 2.5 inches.

13. The device of claim 10, wherein both the first and rollers has a diameter of about 2.25 inches.

14. The device of claim 1, wherein the distance between the first and second roller is not more than 1 inch.

15. The device of claim 1, wherein each of the first and second rollers have an outer surface.

16. The device of claim 15, wherein the outer surface is smooth.

17. The device of claim 15, wherein the outer surface is textured.

18. The device of claim 17, wherein the texture includes rounded protrusions.

19. The device of claim 1, wherein the first and second rollers are capable of vibration.

20. The device of claim 1, wherein the first and second rollers are capable of heating.

21. The device of claim 1, wherein the sleeve is elastic.

22. The device of claim 1, wherein the restrainer comprises first and second seams sewn into the sleeve to define an enclosure.

23. The device of claim 1, wherein the massage device further includes a handle.

24. A kit comprising:
the massage device of claim 1, and
instructional material.

25. The kit of claim 21, wherein the instructional material includes media.

26. The kit of claim 21, wherein the instructional material includes a brochure.

27. The kit of claim 21, wherein the instructional materials include instruction on massage techniques, myofascial release, or trigger point therapy.

28. A method of promoting physical well-being of a body, the method comprising: placing a device comprising first and second rollers between and in contact with the body and a hard surface, wherein the first and second rollers are enclosed within a sleeve and are separated by a distance of less than 1 inch; and
contacting the first and second rollers with the hard surface and the body such that a force is applied to the one or more trigger points or fascia.

29. The method of claim 28, wherein the first and second rollers are touching.

30. The method of claim 28, wherein the surface is a wall or a floor.

31. The method of claim 28, wherein the method is self-administered.

32. The method of claim 28, wherein the rollers are spherical, and the method includes trigger point therapy.

33. The method of claim 28, wherein the rollers are cylindrical and the method includes myofascial release.

34. The method of claim 28, wherein the method alleviates pain.

35. The method of claim 33, wherein the method is a treatment for fibromyalgia.

36. A method for performing a trigger-point massage, comprising:
providing a device including first and second spherical rollers enclosed within a sleeve, and
applying localized pressure to a trigger-point using the first and second rollers, wherein the first and second rollers are spaced apart by a distance of no more than 2 inches.
